(19) United States
(12) Patent Application Publication
(10) Pub. No.: US 2008/0199367 A1
(43) Pub. Date: Aug. 21, 2008

(54) Indoor air freshener

(75) Inventor: Chun-Hui Lin, Taipei City (TW)

Correspondence Address: TROXELL LAW OFFICE PLLC, 5205 LEESBURG PIKE, SUITE 1404, FALLS CHURCH, VA, 22041, US

(73) Assignee: Hocheng Corporation, Taipei (TW)

(21) Appl. No.: 11/716,037

(22) Filed: Mar. 9, 2007

(30) Foreign Application Priority Data
Feb. 15, 2007 (TW) 096203034

(51) Int. Cl. A61L 9/20; A61L 9/00

(52) U.S. Cl. 422/121; 422/120; 422/123

(57) ABSTRACT

An air is dehumidified by a dehumidifier. Then the dehumidified air is heated, sterilized and deodorized at an air outlet.

### FIELD OF THE INVENTION

The present invention relates to an air freshener; more particularly, relates to guiding a dehumidified air to be heated, sterilized and deodorized.

### DESCRIPTION OF THE RELATED ART

In a rainy and wet place, in a cold and wet winter or in a rainy season, a dehumidifier is used to keep room dry, to prevent clothes from mildew and to avoid floor from becoming viscoid.

A general dehumidifier emphasizes on humidity and temperature control; or, together with a design of an automatic turning-on and turning-off.

However, the automatic design only turns on the dehumidifier for dehumidification at a certain time and closes it at another time. Or, the dehumidifier is closed on reaching a certain humidity or temperature, Or, the dehumidifier is closed when the water collector is full. Yet the air outputted from the dehumidifier is not processed and is usually directly outputted. Consequently, after a period of time of use, the dehumidifier may become dirty and contaminated; and the air outputted may be full of dirt and germs and even odor.

Therefore, the indoor air quality becomes terrible. Hence, the prior art does not fulfill users' requests on actual use.

### SUMMARY OF THE INVENTION

The main purpose of the present invention is to heat, sterilize and deodorize a dehumidified air by a heater, an optical catalyst unit and an aromatic unit in coordination with an air outlet of a dehumidifier.

To achieve the above purpose, the present invention is an indoor air freshener, comprising a heater; an optical catalyst unit at a side of the heater; an aromatic unit at a side of the optical catalyst unit; and a power unit connecting to the heater, the optical catalyst unit and the aromatic unit to supply with power, where the heater, the optical catalyst unit and the aromatic unit are located in a dehumidifier. Accordingly, a novel indoor air freshener is obtained.

### BRIEF DESCRIPTIONS OF THE DRAWINGS

The present invention will be better understood from the following detailed descriptions of the preferred embodiments according to the present invention, taken in con junction with the accompanying drawings, in which

FIG. 1 is the block view according to the present invention;

FIG. 2 is the explosive view showing the first preferred embodiment;

FIG. 3 is the sectional view showing the state of use of the first preferred embodiment;

FIG. 4 is the sectional view showing the state of use of the second preferred embodiment;

FIG. 5 is the sectional view showing the state of use of the third preferred embodiment;

FIG. 6 is the perspective view showing the fourth preferred embodiment; and

FIG. 7 is the sectional view showing the state of use of the fourth preferred embodiment.

### DESCRIPTIONS OF THE PREFERRED EMBODIMENTS

The following descriptions of the preferred embodiments are provided to understand the features and the structures of the present invention.

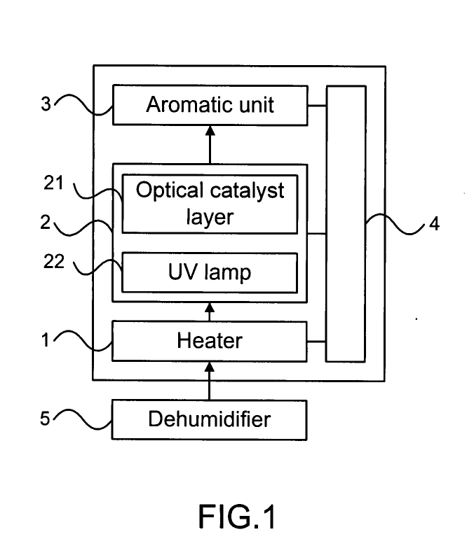

Please refer to FIG. 1, which is a block view according to the present invention. As shown in the figure, the present invention is an indoor air freshener, comprising a heater 1, an optical catalyst unit 2, an aromatic unit 3, a power unit 4 and a dehumidifier 5.

The heater 1 is made of ceramics; or is a heating pipe of coils.

The optical catalyst unit 2 is located at a side of the heater; and comprises an optical catalyst layer 21 and an ultraviolet (UV) lamp 22 corresponding to the optical catalyst layer 21.

The aromatic unit 3 is located at a side of the optical catalyst unit; and has a liquid aromatic or a solid aromatic.

The power unit 4 is connected with the heater 1, the optical catalyst unit 2 and the aromatic unit 3 to supply with power.

Air outputted from the dehumidifier 5 is passed through the heater 1, the optical catalyst unit 2 and the aromatic unit 3 to be heated, sterilized and deodorized. Thus, a novel indoor air freshener is obtained.

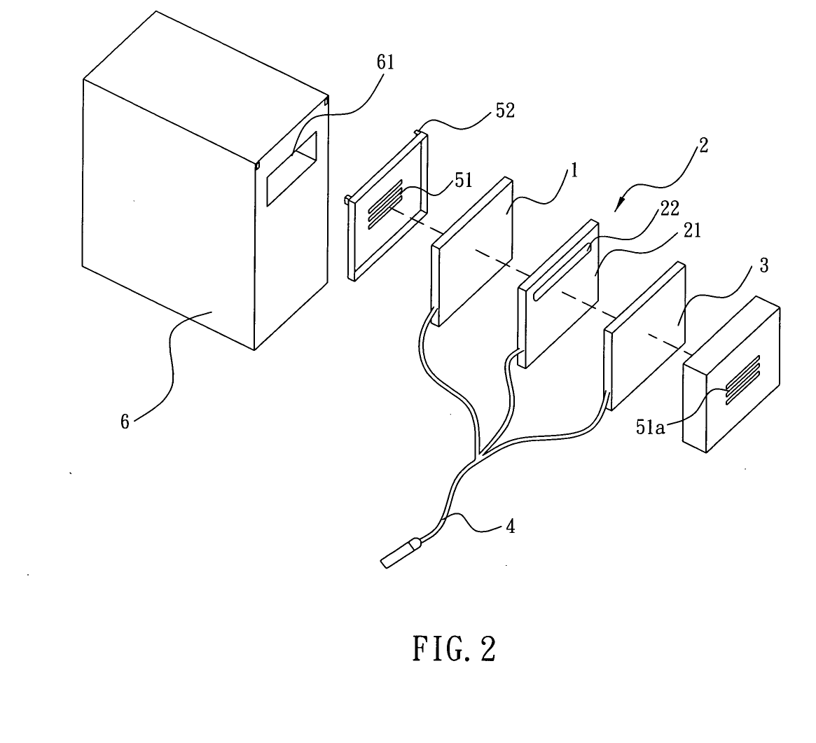

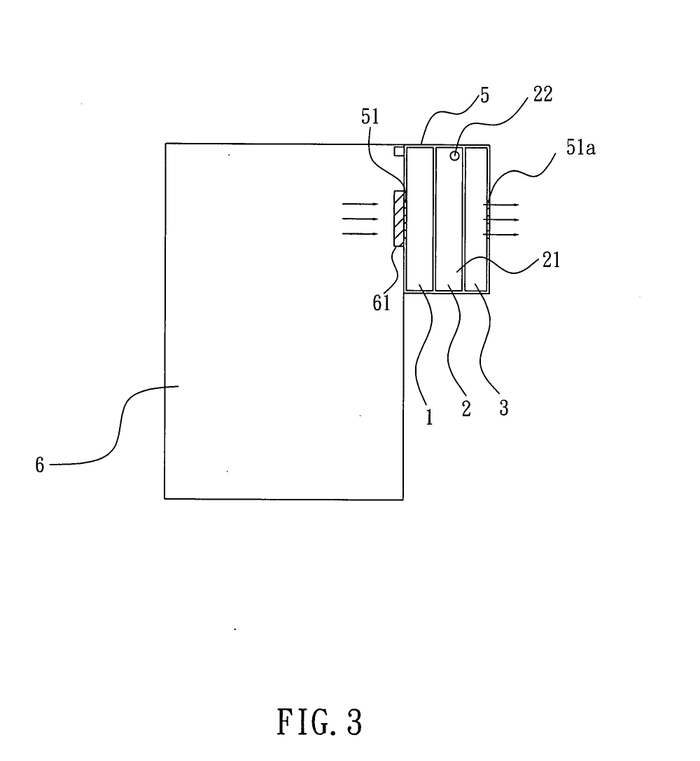

Please refer to FIG. 2 and FIG. 3, which are an explosive view and a sectional view of a state of use showing a first preferred embodiment. As shown in the figures, when using the first preferred embodiment, a heater 1, an optical catalyst unit 2 and an aromatic unit 3 is put in a case 6; and a power unit 4 is deposed outside at a side of the case 6. Each of two opposite surfaces of the case 6 has a plurality of holes 61, 61a; and the case 6 has a plurality of fixing members 62. On assembling the first preferred embodiment, the case 6 is fixed at outside on an air outlet 51 of a dehumidifier 5 by the fixing members 62.

When using the first preferred embodiment, an outside power is supplied to the heater 1, the optical catalyst unit 2 and the aromatic unit 3 by the power unit 4. On running the dehumidifier 5, a dehumidified air is guided to be outputted from the air outlet 51. Then the air enters into the case 6 through the holes 61 on a surface at a side. The air is heated by the heater 1 at first; then is passed through the optical catalyst unit 2. Therein, the heated air is passed through an optical catalyst layer 21 which is irradiated by an ultraviolet (UV) lamp 22 for sterilization. In the end, the heated and sterilized air is passed through the aromatic unit 3 for deodorization to be outputted from the holes 61a on a surface at the opposite side of the case 6. Thus, a dehumidified air from the dehumidifier 5 is guided to be heated, sterilized and deodorized.

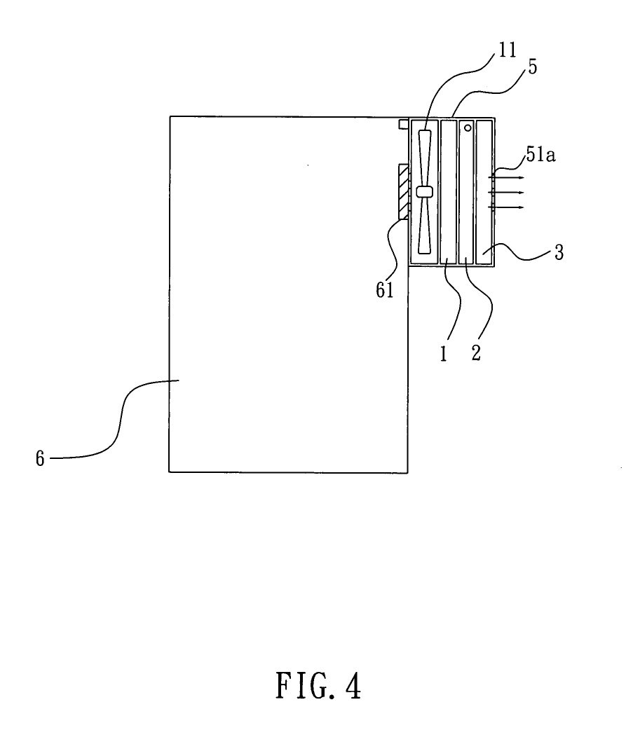

Please refer to FIG. 4, which is a sectional view showing a state of use of a second preferred embodiment. As shown in the figure, on using the second preferred embodiment, an electric fan 11, which is connected with a power unit 4, is applied at a side of a heater 1 to help outputting air from holes 61a on a surface at a side of the case 6.

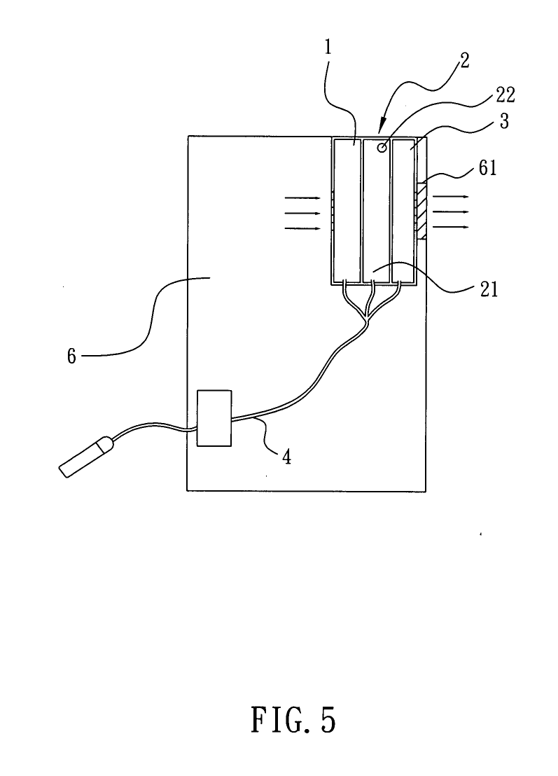

Please refer to FIG. 5, which is a sectional view showing a state of use of a third preferred embodiment. As shown in the figure, on using the third preferred embodiment, a heater 1, an optical catalyst unit 2 and an aromatic unit 3 are put in a dehumidifier 5. The heater 1 is corresponding to an inner side of an air outlet 51; and the aromatic unit 3 is abided on a surface corresponding to an outer side of the air outlet 51. And a power unit 4 is connected with the dehumidifier 5.

On using the third preferred embodiment, after the air dehumidified by the dehumidifier 5 is guided to be heated by the heater 1, the air is passed through the optical catalyst unit 2, where the air is passed through an optical catalyst layer 21 irradiated by an UV lamp 22 for sterilization. In the end, the heated and sterilized air is passed through the aromatic unit 3 for deodorization to be outputted from the air outlet 51 of the dehumidifier 6. Thus, a dehumidified air from the dehumidifier 5 is guided to be heated, sterilized and deodorized.

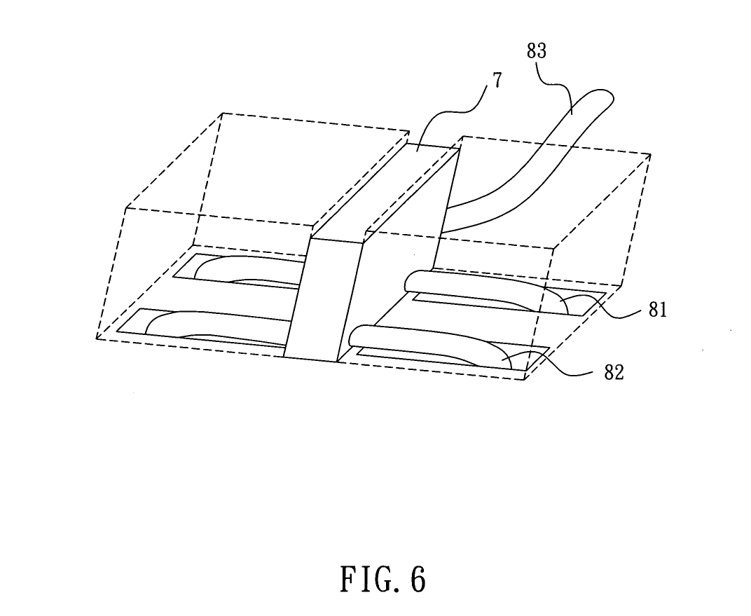

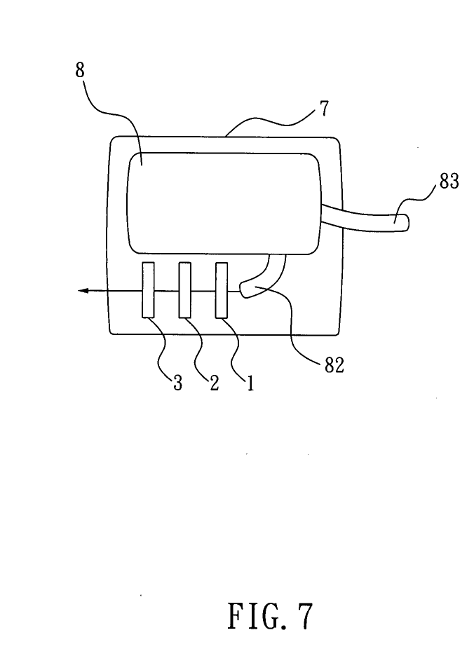

Please refer to FIG. 6 and FIG. 7, which are a perspective view and a sectional view of a state of use showing a fourth preferred embodiment. As shown in the figures, a dehumidifier 8 is put in a shell 7. The dehumidifier 8 has an air-sucking pipe 81 at outside of the case; an air-outlet pipe 82; and a water-outlet pipe 83. A heater 1 is deposed in the shell 7 at a position corresponding to the air-outlet pipe 82; an optical catalyst unit 2 is deposed at a side of the heater 1; and an aromatic unit 3 is deposed at a side of the optical catalyst unit 2.

On using the fourth preferred embodiment, the shell 7 is located between two bathroom sink 9 for hiding. The water-outlet pipe 83 is set at the draining hole in a bathroom. And the air-sucking pipe 81 and the air-outlet pipe 82 are placed in a required place according to a design. On running the fourth preferred embodiment, after the air dehumidified by the dehumidifier 8 is guided to be heated by the heater 1, the air is sterilized through the optical catalyst unit 2. Finally the heated and sterilized air is passed through the aromatic unit 3 for deodorization to be outputted from the air-outlet pipe 82 of the dehumidifier 8. Thus, a dehumidified air from the dehumidifier 8 is guided to be heated, sterilized and deodorized.

To sum up, the present invention san indoor air freshener, wherein coordination with an air outlet of a dehumidifier, a dehumidified air is heated, sterilized and deodorized by a heater, an optical catalyst unit and an aromatic unit respectively.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. An indoor air freshener, comprising:
a heater;
an optical catalyst unit, said optical catalyst unit being deposed at a side of said heater;
an aromatic unit, said aromatic unit being deposed at a side of said optical catalyst unit; and
a power unit, said power unit being connected with said heater, said optical catalyst unit and said aromatic unit to supply said heater, said optical catalyst unit and said aromatic unit with power; and
a dehumidifier,
wherein an air obtained from said dehumidifier is passed through said heater, said optical catalyst unit and said aromatic unit to be heated, sterilized and deodorized.

2. The air freshener according to claim 1,
wherein said heater, said optical catalyst unit and said aromatic unit is located in a case;
wherein said case has a plurality of holes on each of two corresponding sides of said case; and
wherein said case has a plurality of fixing members.

3. The air freshener according to claim 1,
wherein said heater is made of ceramics.

4. The air freshener according to claim 1,
wherein said heater is a heating pipe of a plurality of coils.

5. The air freshener according to claim 1,
wherein an electric fan is deposed at a side of said heater.

6. The air freshener according to claim 1,
wherein said optical catalyst unit comprises an optical catalyst layer; and an ultraviolet (UV) lamp corresponding to said optical catalyst layer.

7. The air freshener according to claim 1,
wherein said aromatic unit has a liquid aromatic.

8. The air-freshener according to claim 1,
wherein said aromatic unit has a solid aromatic.

9. An indoor air freshener, comprising:
a shell;
a dehumidifier, said dehumidifier being deposed in said shell, said dehumidifier having an air-sucking pipe, an air-outlet pipe and a water-outlet pipe, said air-sucking pipe, said air-outlet pipe and said water-outlet pipe being deposed on outer surface of said shell;
a heater, said heater being deposed in said shell, said heater being corresponding to said air-outlet
an optical catalyst unit, said optical catalyst unit being deposed in said shell, said optical catalyst unit being located at a side of said heater; and
an aromatic unit, said aromatic unit being deposed in said shell, said aromatic unit being located at a side of said optical catalyst unit.

10. The air freshener according to claim 9,
wherein said heater is made of ceramics.

11. The air freshener according to claim 9,
wherein said heater is a heating pipe of a plurality of coils.

12. The air freshener according to claim 9,
wherein said optical catalyst unit comprises an optical catalyst layer; and an UV lamp corresponding to said optical catalyst layer.

13. The air freshener according to claim 9,
wherein said aromatic unit has a liquid aromatic.

14. The air freshener according to claim 9,
wherein said aromatic unit has a solid aromatic.
